(19) United States
(12) Patent Application Publication
(10) Pub. No.: US 2025/0095194 A1
(43) Pub. Date: Mar. 20, 2025

(54) IMAGE PROCESSING APPARATUS, STORAGE MEDIUM, AND IMAGE PROCESSING METHOD

(71) Applicant: Konica Minolta, Inc., Tokyo (JP)

(72) Inventors: Ryohei ITO, Tokyo (JP); Amai SHIMIZU, Tokyo (JP); Yasuyuki MORITA, Tokyo (JP); Miki OKAZAKI, Tokyo (JP); Ayako YOSHIDA, Tokyo (JP)

(21) Appl. No.: 18/883,600

(22) Filed: Sep. 12, 2024

(30) Foreign Application Priority Data
Sep. 19, 2023 (JP) 2023-150728

(51) Int. Cl. G06T 7/73; A61B 6/00; A61B 6/50; G16H 30/40

(52) U.S. Cl. CPC G06T 7/73; A61B 6/505; A61B 6/545; G16H 30/40; G06T 2207/10116; G06T 2207/20081; G06T 2207/30008

(57) ABSTRACT

Provided is an image processing apparatus that includes a hardware processor. The hardware processor acquires a two-dimensional radiation image; and analyzes the acquired two-dimensional radiation image to infer three-dimensional structure information on a structure on an inside of a subject.

### CROSS-REFERENCE TO RELATED APPLICATIONS

The entire disclosure of Japanese Patent Application No. 2023-150728, filed on Sep. 19, 2023, including description, claims, drawings and abstract is incorporated herein by reference.

### TECHNICAL FIELD

The present invention relates to an image processing apparatus, a storage medium, and an image processing method.

### BACKGROUND ART

In radiation imaging using radiation such as X-rays performed in a medical facility such as a hospital, when a patient is not in a proper posture for imaging, a misalignment occurs in positioning. In such a case, a radiologist or other personnel grasps the three-dimensional positional relationship of a structure(s) inside the subject while viewing a radiation image previously captured, and guides the patient to an appropriate positioning.

Techniques for capturing a radiation image using three-dimensional information are disclosed in the following documents. JP4484462B2 describes a method of detecting a body region based on an image of a patient captured by a 3D scanner or the like and automatically presenting, on a screen, a scanning range that selectively covers the detected body region. JP4709600B2 describes an X-ray diagnostic apparatus that calculates an arcuated moving path about a blood vessel of interest based on data of a standard three-dimensional model related to a blood vessel at an arbitrary site and supports optimization of an imaging angle.

According to the conventional technique, it is possible to support the positioning of the scanning range and the optimization of the imaging angle with respect to the blood vessel. However, the conventional technique cannot present a three-dimensional positional relationship of a structure(s) inside a subject in a radiation image to a radiologist. Therefore, since there is no information serving as a basis of positioning correction, it is impossible to efficiently determine a direction of positioning correction, problematically.

### SUMMARY OF THE INVENTION

Therefore, an object of the present invention is to provide an image processing apparatus, a storage medium, and an image processing method capable of efficiently determining a direction of positioning correction and the like.

### Solution to Problem

To achieve at least one of the abovementioned objects, according to an aspect of the present invention, an image processing apparatus reflecting one aspect of the present invention includes:
a hardware processor,
wherein the hardware processor acquires a two-dimensional radiation image; and
analyzes the acquired two-dimensional radiation image to infer three-dimensional structure information on a structure on an inside of a subject.

According to an aspect of the present invention, a storage medium reflecting another aspect of the present invention stores a program for causing a computer to function as:
a first acquisition section that acquires a two-dimensional radiation image; and
an inference section that analyzes the acquired two-dimensional radiation image to infer three-dimensional structure information on a structure on an inside of a subject.

According to an aspect of the present invention, an image processing method reflecting another aspect of the present invention includes:
acquiring a two-dimensional radiation image; and
analyzing the acquired two-dimensional radiation image to infer three-dimensional structure information on a structure on an inside of a subject.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a diagram illustrating a schematic configuration of a photographing support system according to the present embodiment;

FIG. 2 is a block diagram of an imaging control device according to the present embodiment;

FIG. 3 is a view showing an example of an arrangement of an imaging support information output table stored in the storage section according to the present embodiment;

FIG. 4A is a diagram for explaining actions of external rotation and internal rotation;

FIG. 4B is a diagram for explaining actions of abduction and adduction;

FIG. 5 is a diagram illustrating an analysis result of a misalignment in positioning in a radiation image of a “knee (right knee) joint lateral side” in the related art;

FIG. 6A is a diagram illustrating an example of input data used for training a machine learning model according to the present embodiment;

FIG. 6B is a diagram illustrating an example of ground truth data used for training a machine learning model according to the present embodiment;

FIG. 7 is a diagram illustrating an example of other ground truth data for training machine learning model according to the present embodiment;

FIG. 8 is a flowchart illustrating an example of operations of the imaging control device at the time of capturing a radiation image of a subject according to the present embodiment;

FIG. 9 is a diagram illustrating an example of a configuration of an imaging screen displayed on the display section according to the present embodiment;

FIG. 10 is a flowchart illustrating an example of operations of the imaging control device at the time of re-imaging determination processing according to the present embodiment;

FIG. 11A is a diagram illustrating an example of three-dimensional structure information of a radiation image inferred from the machine learning model according to the present embodiment;

FIG. 11B is a diagram illustrating an example of three-dimensional structure information of a radiation image inferred from the machine learning model according to the present embodiment;

FIG. 12 is a flowchart illustrating an example of the first determination processing according to the present embodiment;

FIG. 13 is a flowchart illustrating an example of the second determination processing according to the present embodiment;

FIG. 14 is a diagram illustrating an example of imaging support information displayed on the imaging screen according to the present embodiment;

FIG. 15 is a diagram illustrating an example of the imaging screen when text information is displayed in addition to each line information as the three-dimensional structure information;

FIG. 16 is a diagram illustrating an example of the imaging screen when only the medial condyle side line information is displayed as the three-dimensional structure information; and

FIG. 17 is a diagram illustrating a schematic configuration of an imaging support system according to the other Embodiment 1.

### DETAILED DESCRIPTION

In the following, preferred embodiments of the present disclosure will be described with reference to the accompanying drawings.

### [Configuration Example of Imaging Support System 10A]

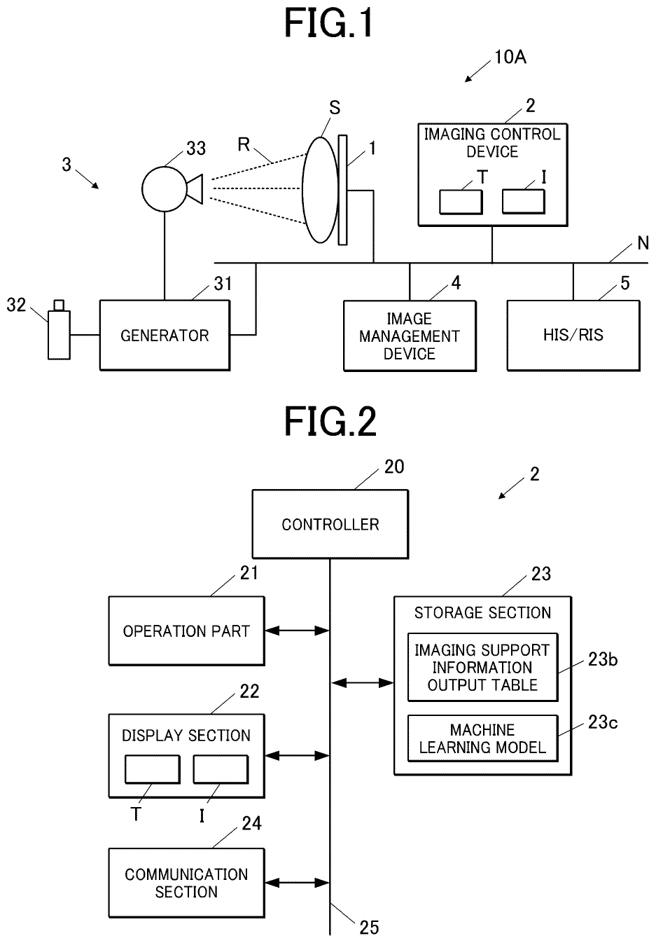

FIG. 1 is a diagram illustrating a schematic configuration of an imaging support system 10A according to the present embodiment. The imaging support system 10A includes a radiographic imaging device 1, an imaging control device 2, a radiation generating device 3, an image management device 4, and a HIS/RIS 5. Hereinafter, the radiographic imaging device may be referred to as the imaging device 1, and the radiation generating device may be referred to as the generation device 3. HIS is an abbreviation for Hospital Information System and is a hospital information system. RIS is an abbreviation for Radiology Information System and is a radiology department information system. The radiation image is an example of a medical image. The imaging control device 2 is an example of an image processing apparatus.

The imaging device 1, the imaging control device 2, the generation device 3, the image management device 4, and the HIS/RIS 5 are communicably connected to each other via a network N. Examples of the network N include a LAN, a WAN, or the Internet. LAN is an abbreviation for Local Area Network. WAN is an abbreviation for Wide Area Network. A communication system of the network N may be wired communication or wireless communication. The wireless communication includes, for example, Wi-Fi®.

The generation apparatus 3 includes a generator 31, a switch 32, and a radiation source 33. The generator 31 applies, to the radiation source 33 including, for example, a tube, a voltage according to imaging conditions set in advance, in response to operation of the switch 32. The generator 31 may include an operation part that receives input of irradiation conditions and the like.

When the generator 31 applies a voltage, the radiation source 33 generates radiation R having a dose according to the applied voltage. The radiation R is, for example, X-rays.

The generation device 3 generates the radiation R in a manner corresponding to the type of radiation image, for example, a still image or a dynamic image. To be specific, in generating a still image, the generation device 3 emits the radiation R only once in response to the switch 32 being pressed once. In generating a dynamic image, for example, in response to the switch 32 being pressed once, the generation device 3 repeatedly emits pulsed radiation R multiple times per predetermined period of time.

The imaging device 1 generates digital image data in which an imaging site of the subject S is captured. For the imaging device 1, for example, a portable FPD is used. FPD is an abbreviation for Flat Panel Detector. The imaging device 1 may be integrated with the generation device 3.

Although not illustrated, the imaging device 1 includes, for example, an imaging element, a sensor substrate, a scanner, a reader, a controller, and a communicator. The imaging element generates electric charges according to the dose of received radiation R. The switch elements are two-dimensionally arranged in the sensor substrate, and the sensor substrate accumulates and discharges electric charge. The scanner switches on and off of each switch element. The reader reads, as a signal value, an amount of electric charge emitted from each pixel. The controller generates image data of a radiation image from the plurality of signal values read by the reading unit. The image data includes still image data or dynamic image data. The communication section transmits the generated image data, various signals, and the like to other devices such as the imaging control device 2, and receives various kinds of information and various signals from other devices.

The imaging control device 2 sets imaging conditions for the imaging device 1, the generation device 3, and the like, and controls a reading operation of the radiation image captured by the imaging device 1. The imaging control device 2 is also called a console, and is constituted by, for example, a personal computer or the like. The imaging control device 2 determines whether or not re-imaging is necessary according to positioning misalignment of the radiation image obtained by imaging. Here, the positioning means, for example, how to position a patient posture during imaging. When determining that re-imaging is necessary, the imaging control device 2 causes a display part 22 (described later) to display the imaging support information I and the three-dimensional structure information T on its screen. The imaging support information I assists positioning correction by presenting a direction in which the positioning is to be corrected (direction of positioning correction) or the like in the form of words or sentences. The three-dimensional structure information T is information indicating a three-dimensional positional relationship of a structure(s) inside a subject in a radiation image, and presents information serving as a basis for correcting positioning.

The imaging conditions include, for example, patient conditions related to a subject S, irradiation conditions related to the emission of the radiation R, and image reading conditions related to the image reading of the imaging device 1. The patient conditions include, for example, an imaging site, an imaging direction, and a physique. The irradiation conditions are, for example, tube voltage (kV), tube current (mA), irradiation time (ms), current-time product (mAs value), and the like. The image reading conditions include, for example, a pixel size, an image size, and a frame rate. The imaging control device 2 may automatically set the imaging conditions on the basis of the order information acquired from the HIS/RIS 5 or the like. The imaging control device 2 may set the imaging conditions in response to manual operations by a user such as a radiologist on an operation part 21 described later.

The image management device 4 manages the image data generated by the imaging device 1. The image management device 4 is a picture archiving and communication system, a diagnostic imaging workstation, or the like. The picture archiving and communication system may be referred to as PACS. PACS is an abbreviation for Picture Archiving and Communication System.

The HIS/RIS 5 receives, for example, the order information on the radiographing of the patient from a doctor or the like, and transmits the received order information to the imaging control device 2. The order information includes, for example, various kinds of information such as an ID, an imaging site, an imaging direction, and a body type of the patient.

### [Example of Configuration of Block Diagram of Imaging Control Device 2]

FIG. 2 is a block diagram of the imaging control device 2. The imaging control device 2 includes a controller 20 (hardware processor), the operation part 21, the display part 22, a storage section 23, and a communicator 24. The controller 20, the operation part 21, the display part 22, the storage section 23, and the communicator 24 are communicably connected via, for example, a bus 25.

The controller 20 includes, for example, a processor such as a CPU. CPU is an abbreviation for Central Processing Unit.

The processor implements various kinds of processing including imaging control and re-imaging determination by executing various kinds of programs stored in a memory such as a RAM (which may be the storage section 23).

The controller 20 may include an electronic circuit such as an ASIC or an FPGA. ASIC is an abbreviation for Application Specific Integrated Circuit. FPGA is an abbreviation for Field Programmable Gate Array.

The operation part 21 receives a command according to various input operations from a user, converts the received command into an operation signal, and outputs the operation signal to the controller 20. The operation part 21 includes, for example, a mouse, a keyboard, a switch, and a button. The operation part 21 may be, for example, a touch screen integrally combined with a display. The operation part 21 may be, for example, a user interface such as a microphone that receives a voice input.

The display part 22 displays a radiation image based on image data received from the imaging device 1, a GUI for receiving various input operations from the user, and the like. GUI is an abbreviation for Graphical User Interface.

The display part 22 is, for example, a display such as a liquid crystal display or an organic EL display. EL is an abbreviation for Electro Luminescence. Specifically, the display part 22 displays the radiation image obtained by imaging of the imaging device 1, and displays the imaging support information I and the three-dimensional structure information T according to the result of the re-imaging determination processing.

The storage section 23 stores, for example, a system program, an application program, and various types of data.

The storage section 23 includes, for example, any storage module such as an HDD, an SSD, a ROM, and a RAM. HDD is an abbreviation for Hard Disk Drive. SSD is an abbreviation for Solid State Drive. ROM is an abbreviation for Read Only Memory. To be specific, the storage section 23 stores an imaging support information output table 23b and a machine learning model (learned model) 23c. The machine learning model 23c and the like may be stored in an externally provided storage device or the like. Details of the imaging support information output table 23b and the machine learning model 23c will be described later.

The communicator 24 includes, for example, a communication module including an NIC, a receiver, and a transmitter. NIC is an abbreviation for Network Interface Card. The communicator 24 communicates various types of data such as image data among the imaging device 1, the image management device 4, and the like via the network N.

In the present embodiment, the controller 20 (hardware processor) functions as a first acquisition section (acquisition step), an extraction section (extraction step), an inference section, and an output section. Each function of the first acquisition section, the extraction section, the inference section, and the output section is realized by the processor of the controller 20 executing a program stored in the storage section 23 or the like.

The first acquisition section acquires a two-dimensional radiation image captured by the imaging device 1. The controller 20 may function as a second acquisition section that obtains the imaging site information from order information or the like transmitted from a HIS/RIS 5 or the like. The imaging site information can be used in changing parameters or algorithms of a machine learning model (described later). The inference section extracts a structure(s) inside the subject from the two-dimensional radiation image based on the imaging site information acquired from the second acquisition section.

The inference section analyzes the two-dimensional radiation image acquired by the first acquisition section, and infers three-dimensional structure information T on the structures inside the subject of the radiation image. In the present embodiment, it is assumed that the structures inside the object include at least a first structure located on the near side and a second structure located on the far side. The near side is the side of the generation device 3 such as the radiation source 33, and the far side is the side of the imaging device 1. The inference section may infer the three-dimensional structure information T of the structures inside the subject of the radiation image using the machine learning model 23c trained in advance. In this case, by inputting the acquired two-dimensional radiation image to the machine learning model 23c, the inference section can distinguish the two structures inside the subject in the radiation image into the first structure located on the near side and the second structure located on the far side. Details of the three-dimensional structure information T of the structures using the machine learning model 23c will be described later.

Note that the three-dimensional structure information T of the structure inside the subject can also be inferred without using the machine learning model 23c. In that case, the controller 20 (hardware processor) functions as the extraction section and the inference section. Each function of the extraction section and the inference section is implemented by the processor of the controller 20 executing a program stored in the storage section 23 or the like. Specifically, the extraction section and the inference section may infer the three-dimensional structure information T on the structures inside the subject by executing a technique involving structure recognition through edge detection, which is image processing and structure inference through histogram analysis. The extraction section and the inference section may infer the three-dimensional structure information T of the structures inside the subject by executing a technique such as structure recognition by comparison with a correct image by pattern matching.

The output section outputs the three-dimensional structure information T of the structures inside the subject inferred by the inference section of the radiation image. For example, the output section performs output control to display the inferred three-dimensional structure information T of the structures superimposed on the radiation image displayed on the display part. The output section functions as a re-imaging support information output section, and outputs re-imaging support information based on the three-dimensional structure information T inferred by the inference section. The re-imaging support information includes imaging support information I for changing the position of the subject S or the imaging device 1 at the time of re-imaging.

Note that the imaging control device 2 may be configured not to include the operation part 21 and the display part 22. In that case, the imaging control device 2 may receive control signals from an operation part provided in an external device connected via the communicator 24. The imaging control device 2 may output an image signal to a display part provided in the external device to display a radiograph or the like. The external device may be the image management apparatus 4 or the like, or may be another device.

[Example of Configuration of Imaging Support Information Output Table 23b]

Next, an example of the configuration of the imaging support information output table 23b stored in the storage section 23 will be described.

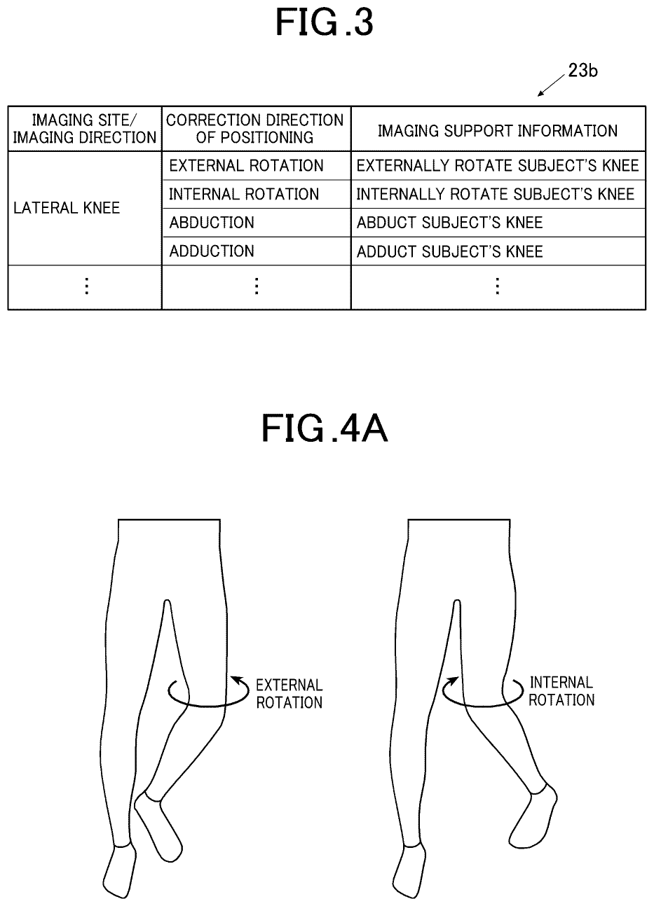

FIG. 3 shows an example of the configuration of the imaging support information output table 23b. The imaging support information output table 23b stores an imaging site, a direction of correction for correcting a positioning misalignment, and imaging support information I for presenting information as the basis of the direction of correction in association with each other. Examples of the imaging site include a “knee joint lateral side”, a “leg lateral side”, and an “elbow lateral side”.

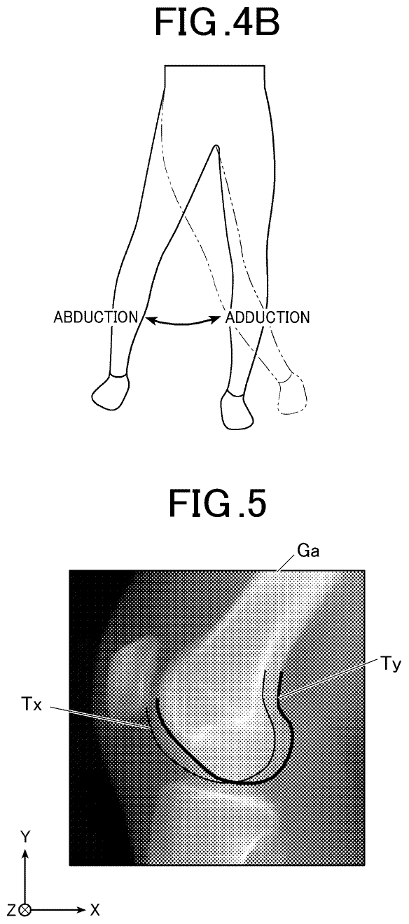

Examples of the type of direction of correction in the case of correcting a positioning misalignment include “external rotation”, “internal rotation”, “abduction”, and “adduction”. FIG. 4A is a diagram for explaining actions of external rotation and internal rotation. As illustrated on the left side of FIG. 4A, external rotation is a movement of rotating the femur outward with respect to the long axis of the bone. Internal rotation is, as illustrated on the right side of FIG. 4A, a movement of rotating the femur inward with respect to the long axis of the bone. FIG. 4B is a diagram for explaining actions of abduction and adduction. Abduction is a movement of the femur away from the central axis of the body. Adduction is a movement of the femur toward the central axis of the body.

Specifically, in a case where the imaging site is “knee joint lateral side” and the direction of positioning correction is “external rotation”, imaging support information I, for example, “please externally rotate your knee” is associated therewith.

In a case where the imaging site is “lateral side of knee joint” and the direction of positioning correction is “internal rotation”, imaging support information I, for example, “please internally rotate your knee” is associated therewith.

When the imaging region is “lateral side of knee joint” and the direction of positioning correction is “abduction”, for example, imaging support information I “please abduct your knee” is associated therewith.

In a case where the imaging site is “lateral side of knee joint” and the direction of positioning correction is “adduction”, for example, imaging support information I “please adduct your knee” is associated therewith.

### [About Machine Learning]

FIG. 5 shows an analysis result of a positioning misalignment in a radiation image Ga of a “lateral side of right knee joint” in the related art. In FIG. 5, the horizontal direction (right-left direction) of the radiation image Ga is defined as the X direction, and the vertical direction (up-down direction) of the radiation image Ga is defined as the Y direction. In addition, an irradiation direction of the radiation R which is a direction orthogonal to the X direction and the Y direction of the radiation image Ga is set as a Z direction. In addition, it is assumed that the orthogonal coordinate system including the X direction, the Y direction, and the Z direction described above is applied to the radiation images, an imaging screen 80 on which the radiation images are displayed, and the like (described below).

In a case where there is a positioning misalignment in the radiation image Ga of the “lateral side of right knee joint”, a misalignment occurs at the medial condyle and the lateral condyle which are respectively positioned in the Z direction of the epiphysis, a structure inside the subject. In this case, since the radiation image Ga is two-dimensionally formed, a line indicating the medial condyle and a line indicating the lateral condyle of the “femoral condyle portion” are displayed on the same plane. Therefore, as shown in FIG. 5, the medial condyle and the lateral condyle are displayed by two lines in the “femoral condyle portion” of the radiation image Ga. These two lines are referred to as a first bone end Tx and a second bone end Ty. In the image analysis in the related art, it is not possible to specify which of the first bone end Tx and the second bone end Ty is a line indicating the medial condyle side positioned on the near side or a line indicating the lateral condyle side positioned on the far side. That is, the three-dimensional positional relationship between the two lines of the first and second bone ends Tx and Ty in the Z direction cannot be specified.

In the present embodiment, the machine learning model 23c is trained by machine learning using machine learning data by a learning device. The learning device is constituted by a computer, for example, and includes a processor such as a CPU and a GPU. GPU is an abbreviation for Graphics Processing Unit. The processor implements a predetermined machine learning function by executing a program stored in the memory such as the RAM, for example. The learning device may be a client device or a server device.

The machine learning model 23c outputs, as inference data, each of a line on the medial condyle side and a line on the lateral condyle side of the “femoral condyle portion” in the radiation image Ga illustrated in FIG. 5. The machine learning model 23c may be, for example, a regression model, such as polynomial regression, multiple regression, support vector regression, or random forest regression. Further, the machine learning model 23c may be another model such as a neural network.

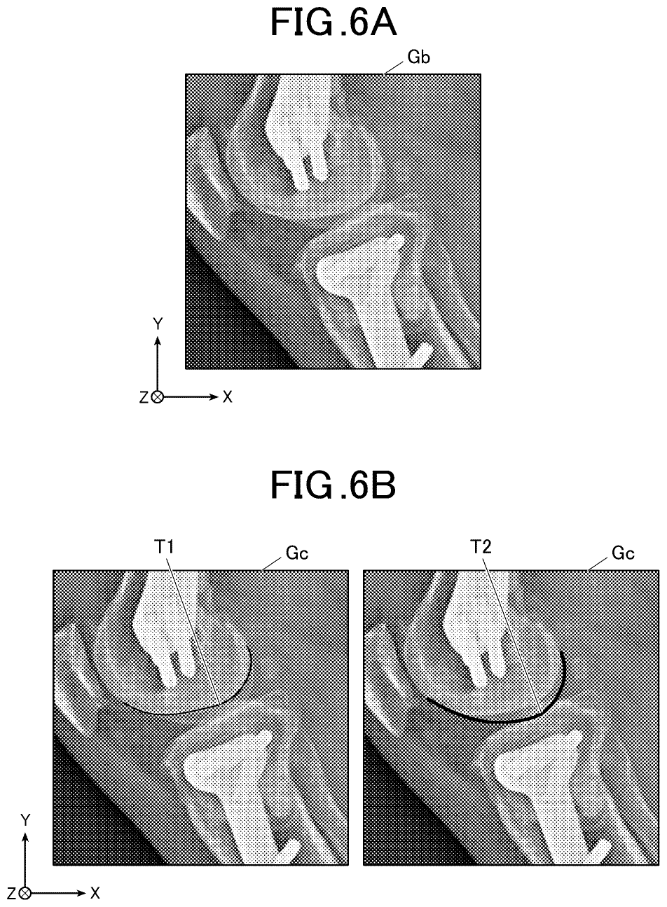

FIG. 6A illustrates an example of input data Gb used to train the machine learning model 23c. FIG. 6B illustrates an example of ground truth data Gc used for training the machine learning model 23c.

The machine learning data includes input data Gb to be input to the machine learning model 23c and ground truth data Gc to be output from the machine learning model 23c. For example, as shown in FIG. 6A, the input data Gb is a radiation image data of the “lateral side of knee joint” in a case where the outer side of the right knee of the patient is imaged in contact with the imaging device 1. The input data Gb has a positioning misalignment in, for example, the “femoral condyle portion”. As the input data Gb, a radiation image of the patient obtained by actual imaging in the past may be used.

As shown in FIG. 6B, the ground truth data Gc is the radiation image data of the “lateral side of knee-joint” in a case where the outer side of the right knee of the patient is imaged in contact with the imaging device 1. The ground truth data Gc includes information T1 on a medial condyle side line indicating a medial condyle area and information T2 on a lateral condyle side line indicating a lateral condyle area, which are correct answers for the input data Gb. Here, the medial condyle side line is a boundary between the medial condyle side of the femoral condyle portion and the soft tissue portion. The lateral condyle line is the boundary between the lateral condyle side of the femoral condyle portion and the soft tissue portion. Hereinafter, the information T1 on the medial condyle side line is referred to as medial condyle side line information T1, and the information T2 on the lateral condyle side line is referred to as lateral condyle side line information T2. The ground truth data Gc may be created by, for example, a user such as a radiologist. The user may specify the medial condyle side line information T1 and the lateral condyle side line information T2 from the radiation image, and tag the line information thereto. The labeled ground truth data Gc can be generated. Note that the medial condyle side line information T1 and the lateral condyle side line information T2 according to the present embodiment correspond to the three-dimensional structure information T.

The learning device performs machine learning using a data set including the input data Gb and the ground truth data Gc described above, and creates a trained machine learning model 23c. When the input data Gb of the “lateral side of right knee joint” is input, the machine learning model 23c outputs the medial condyle side line information T1 and the lateral condyle side line information T2 which are correct in a case where there is a positioning misalignment of the “femoral condyle portion”. That is, the medial condyle and the lateral condyle of the “femoral condyle portion” which is the epiphysis are distinguished into the medial condyle side line information T1 located on the near side and the lateral condyle side line information T2 located on the far side. The trained machine learning model 23c is stored in, for example, the storage section 23 of the imaging control device 2. The imaging control device 2 can identify the type of the positioning misalignment based on the medial condyle side line information T1 and the lateral condyle side line information T2 output from the machine learning model 23c. In this case, the imaging control device 2 can specify whether the “femoral condyle portion” is internally rotated or externally rotated as the type of the misalignment in positioning.

The medial condyle of the femoral condyle portion and the positional relationship in the Z direction of the medial condyle are learned using the medial condyle side line information T1 or the like actually designated by the user, but the learning method is not limited thereto. For example, as another learning method, respective coordinate points of the inner condyle and the outer condyle of the femoral condyle portion may be extracted, and the positional relationship between the medial condyle and the lateral condyle in the Z-axis direction may be inferred by regression or the like of the extracted consecutive coordinate points.

Next, a case of specifying whether the “femoral condyle portion” is adducted or abducted as the type of the positioning misalignment will be described. In this case, the machine learning model 23c is trained by machine learning using the ground truth data Gd different from the ground truth data Gc illustrated in FIG. 6B.

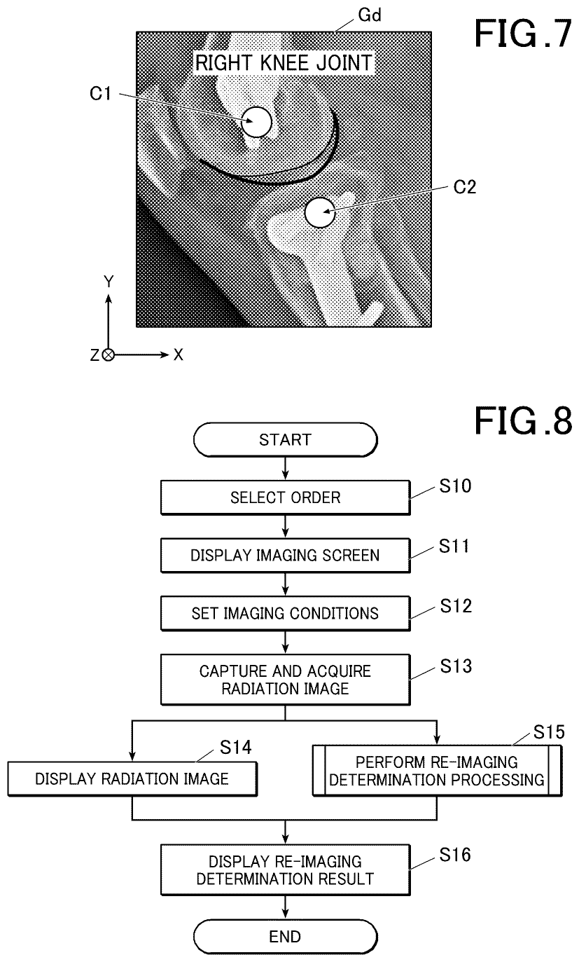

FIG. 7 illustrates an example of another ground truth data Gd used when the machine learning model 23c is trained. Note that the radiation image illustrated in FIG. 6A can be used as the input data Gb, and thus detailed description thereof will be omitted.

The machine learning data includes input data Gb to be input to the machine learning model 23c and ground truth data Gd indicating correct answers for the output of the machine learning model 23c. As illustrated in FIG. 7, the ground truth data Gd includes information C1 indicating the center of femoral condyle and information C2 indicating the center of crural condyle which are correct answers for the input datum Gb. Hereinafter, the information C1 indicating the center of femoral condyle is referred to as femoral condyle center information C1, and the information C2 indicating the center of crural condyle is referred to as crural condyle center information C2. In addition, the ground truth data Gd includes joint information for specifying whether the radiation image of the input data Gb is a right knee joint or a left knee joint. The ground truth data Gd may be created by, for example, a user such as a radiologist. For example, the coordinates of the femoral condyle center information C1 and the coordinates of the crural condyle center information C2, which are the correct “femoral condyle portion”, are determined by visual observation or the like by the user. Subsequently, two heat maps are created in accordance with the determined accuracies of the coordinates of the femoral condyle center information C1 and the coordinates of the crural condyle center information C2. The created heat maps are used as the ground truth data Gd.

When the input data Gb of the “lateral side of knee joint” is input, the machine learning model 23c infers the femoral condyle center information C1, the crural condyle center information C2, and the joint information indicating which of the left and right knee joints is imaged. The trained machine learning model 23c is stored in, for example, the storage section 23 of the imaging control device 2. The imaging control device 2 identifies the type of positioning misalignment based on the femoral condyle center information C1 and the like output from the machine learning model 23c. In this case, the imaging control device 2 can specify whether the “femoral condyle portion” is adducted or abducted as the type of the positioning misalignment.

Note that although the femoral condyle center information C1 and the like of the femoral condyle portion are learned using the heat maps, a method other than this learning method may be used. As another learning method, learning may be performed using the center coordinates themselves of the femoral condyle portion. Further, although the knee joint is specified between two alternatives of the right knee and the left knee, the knee joint may be specified by using other information such as a positional relationship between the femur and the patella.

Alternatively, one machine learning model 23c may be used to infer all of the above-described medial condyle side line information T1, femoral condyle center information C1, and the like. Alternatively, a plurality of machine learning models 23c may be used. In that case, the medial condyle side line information T1 and the like may be inferred with one machine learning model 23c, and the femoral condyle center information C1 and the like may be inferred with another machine learning model 23c.

In the above-described example, the three-dimensional structures of the femoral condyle portion on the “lateral side f right knee joint” in a case where the outer side of the right knee of the patient is imaged in contact with the imaging device 1 is learned, but the present invention is not limited thereto. For example, the three-dimensional structures of the femoral condyle portion on the “lateral side of right knee joint” may be learned by using the radiation image of the “right knee joint side surface” in a case where the inner side of the right knee of the patient is imaged in contact with the imaging device 1. In that case, the medial condyle side line information T1 is positioned on the far side in the Z direction, and the lateral condyle side line information T2 is positioned on the near side in the Z direction. Further, the imaging site targeted for machine learning may be a region other than the “lateral side of right knee joint”. For example, the imaging site targeted for machine learning may be the lateral side of left knee joint, or may be another part such as the ankle joint or elbow joint.

### [Operation Example of Imaging Control Device 2]

Next, a flow in a case where a radiation image of the subject S is captured will be described. FIG. 8 is a flowchart illustrating an example of operations of the imaging control device 2 at the time of capturing a radiation image of the subject S. Hereinafter, a case in which the “lateral side of knee (right knee) joint” is imaged as the imaging site and the imaging direction of the radiation image will be described.

The communicator 24 of the imaging control device 2 receives the order information transmitted from the HIS/RIS 5 or the like. The user selects, for example, predetermined order information from the examination list displayed on the screen of the display part 22 of the imaging control device 2. The controller 20 acquires the order information selected by the user (step S10).

Upon acquiring the predetermined order information, the controller 20 allows the display part 22 to display the imaging screen 80 (step S11).

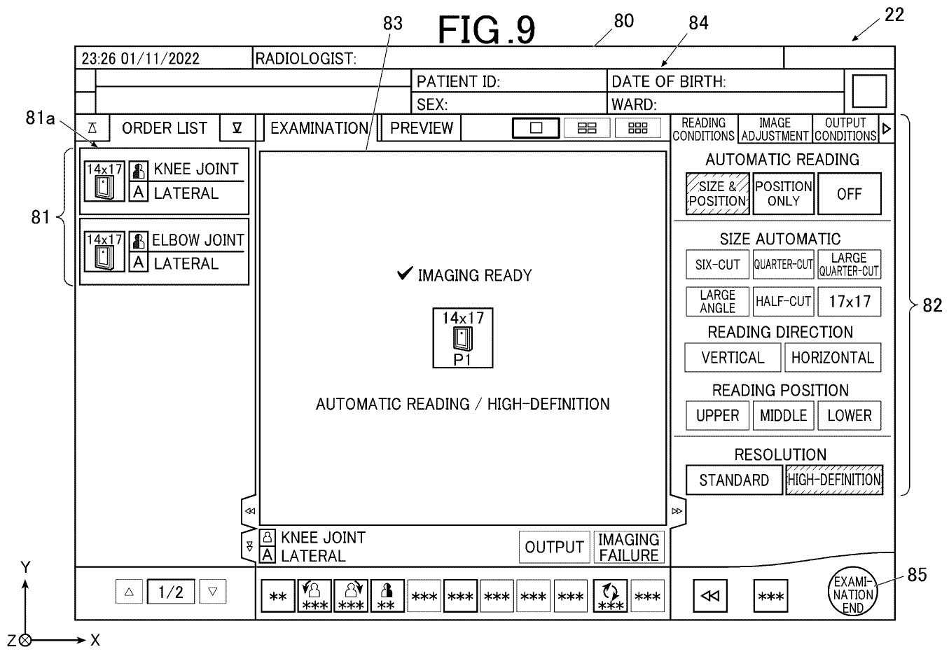

FIG. 9 shows an example of a configuration of an imaging screen 80 displayed on the display part 22 of the imaging control device 2. The imaging screen 80 is provided with an imaging selection area 81, a condition setting area 82, an image display area 83, a patient information display area 84, and an examination end button 85. In the imaging selection area 81, for example, the pieces of order information selected from the examination list are displayed. The order information includes, for example, imaging contents such as an imaging site and an imaging direction, and a thumbnail image indicating a captured radiation image. The condition setting area 82 is provided with, for example, a button for setting imaging conditions of a radiation image, a button for performing image adjustment of a captured radiation image, and the like. In the image display area 83, a radiation image captured by the imaging device 1 is displayed on the basis of the set imaging conditions or the like. Note that in FIG. 9, no radiation image is displayed in the image display area 83 because no radiation image has been captured.

When predetermined order information is selected in the imaging selection area 81 or the like, the controller 20 sets imaging conditions in each of the imaging device 1 and the generation device 3 (Step S12). The imaging conditions include image reading conditions to be set for the imaging device 1 and irradiation conditions to be set for the generation device 3. For example, the controller 20 sets the image reading conditions for the imaging device 1 on the basis of the imaging site, the imaging direction, and the like of the selected order information. Further, the controller 20 sets an irradiation condition in the generation device 3 based on the imaging site, the imaging direction, and the like of the selected order information.

The imaging conditions may be manually set by the user. Specifically, the controller 20 may set the image reading condition received by the input operation in the condition setting area 82 by the user in the imaging device 1. The controller 20 may set the radiation irradiation conditions received by user's input operation on the operation panel of the generation device 3 for the generation device 3.

Subsequently, when the switch 32 is turned on by the user, the controller 20 controls the imaging device 1, the generation device 3, and the like to capture a radiation image of the subject S (Step S13). The generation device 3 emits the radiation R to the imaging site of the subject S. The imaging device 1 detects the radiation R transmitted through the subject S from the generation device 3, and generates image data including the imaging site on the basis of the detected radiation R. The imaging device 1 transmits the generated image data to the imaging control device 2. The controller 20 acquires the radiation image based on the image data transmitted from the imaging device 1 (step S13).

Upon completion of Step S13, the process branches to Step S14 and Step S15. In the present embodiment, an example in which the processing of Step S14 and the processing of Step S15 are performed in parallel will be described, but the present invention is not limited thereto. For example, serial processing in which the processing of Step S14 and the processing of Step S15 are performed in order may be adopted. In this case, Step S15 may be performed first, and Step S14 and Step S16 which will be described later may be performed in combination.

First, the processing of Step S14 will be described.

The controller 20 causes the acquired radiation image to be displayed in the image display area 83 of the imaging screen 80 (Step S14). In the present embodiment, the radiation image of a “lateral side of right knee joint” as the imaging site or the like is displayed in the image display area 83. In the order information 81a of the imaging selection area 81, a thumbnail image representing the captured radiation image is displayed. Upon completion of Step S14, the process proceeds to Step S16.

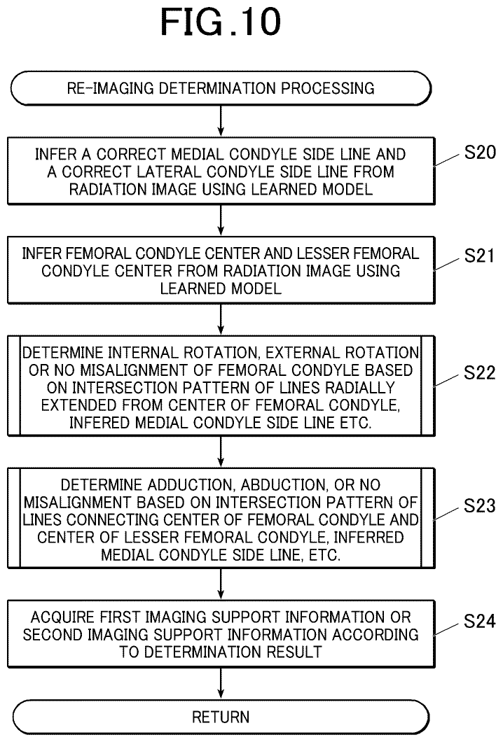

Subsequently, the processing in Step S15 which is branched from Step S13 will be described. The controller 20 executes re-imaging determination processing for determining whether or not re-imaging is necessary using the acquired radiation image. The controller 20 proceeds to the subroutine illustrated in FIG. 10.

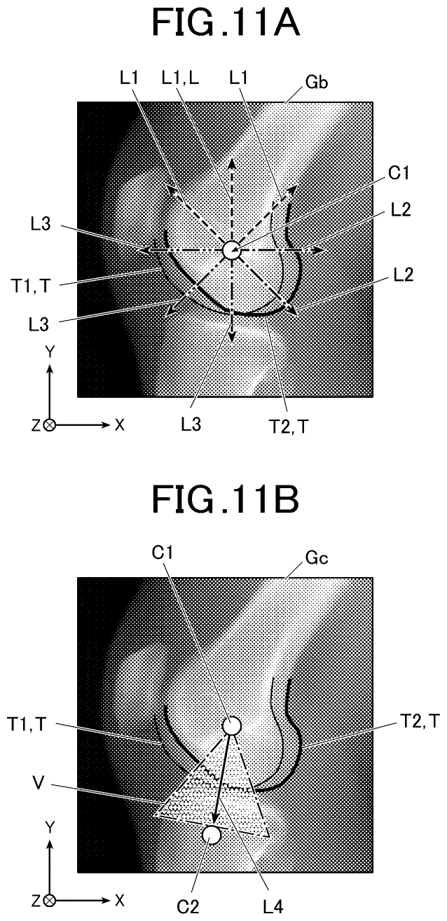

FIG. 10 is a flowchart illustrating an example of the operations of the imaging control device 2 during the re-imaging determination processing. FIGS. 11A and 11B illustrate examples of the three-dimensional structure information T on the femoral condyle portion in the radiation image G inferred by the machine learning model 23c.

Using the machine learning model 23c, the controller 20 infers the medial condyle side line information T1 and the lateral condyle side line information T2 on the femoral condyle portion from the radiation image G obtained by imaging (Step S20). To be specific, the controller 20 inputs the radiation image G of the “lateral side of knee joint” to the machine learning model 23c. Based on the input radiation image G, the machine learning model 23c outputs correct medial condyle side line information T1 and correct lateral condyle side line information T2 of the femoral condyle portion. In the drawing 11A, the medial condyle side line information T1 is indicated by a thin line, and the lateral condyle side line information T2 is indicated by a thick line. In this way, the controller 20 acquires the three-dimensional structure information T divided into the medial condyle side line information T1 positioned on the near side and the lateral condyle side line information T2 positioned on the far side.

Subsequently, the controller 20 uses the machine learning model 23c to infer the femoral condyle center information C1, the crural condyle center information C2, and the joint information from the radiation image G obtained by imaging (Step S21). To be specific, the controller 20 inputs the radiation image of the “lateral side of knee joint” to the machine learning model 23c. Based on the input radiation image G, the machine learning model 23c outputs femoral condyle center information C1 and crural condyle center information C2 of the femoral condyle as inferred data. Furthermore, based on the input radiation image G, the machine learning model 23c outputs joint information indicating that the radiation image G captures the right knee joint. In this way, the controller 20 acquires the femoral condyle center information C1, the crural condyle center information C2, and the joint information. Note that the joint information may be acquired from inspection order information transmitted from the HIS/RIS 5.

In the present embodiment, Step S20 and Step S21 have been described separately, but Step S20 and Step S21 may be one step. To be specific, the machine learning model 23c may output all of the medial condyle side line information T1, the lateral condyle side line information T2, the femoral condyle center information C1, the crural condyle center information C2, and the joint information on the basis of the input radiation image G. When Step S21 is completed, the process proceeds to Step S22.

The controller 20 determines whether the misalignment is internal rotation or external rotation based on an intersection pattern between a line radially extending from the femoral condyle center information C1 and the medial condyle side line information T1 or the like (Step S22). In the present embodiment, processing of determining whether the misalignment of the positioning is internal rotation or external rotation is referred to as first determination processing.

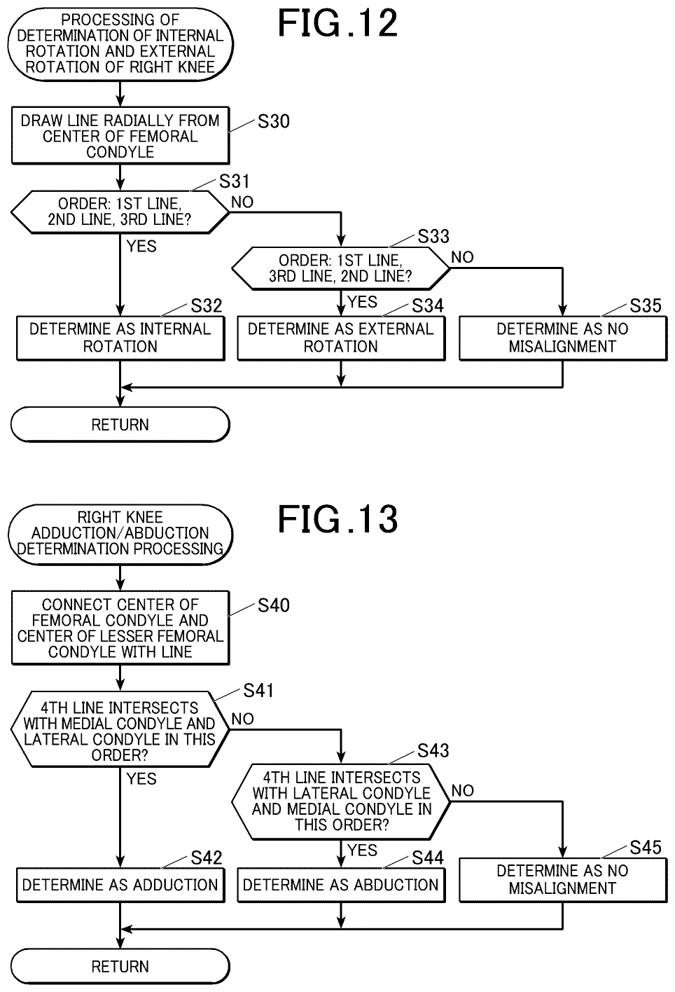

The controller 20 shifts to the subroutine shown in FIG. 12 and functions as the first determiner to execute the first determination processing. FIG. 12 is a flowchart illustrating an example of the first determination processing.

The controller 20 extends a plurality of lines L radially from the inferred femoral condyle center information C1 (Step S30). To be specific, as shown in FIG. 11A, six lines L are radially extended at equal intervals in the circumferential direction from the femoral condyle center information C1. A line L that does not intersect with any of the medial condyle side line information T1 and the lateral condyle side line information T2 is referred to as a first line L1. In FIG. 11A, the first line L1 is indicated by a broken line. A line L that crosses the medial condyle side line information T1 and the lateral condyle side line information T2 in this order is referred to as a second line L2. In FIG. 11A, the second line L2 is indicated by a dash dot line. A line L that crosses the lateral condyle side line information T2 and the medial condyle side line information T1 in this order is referred to as a third line L3. In FIG. 11A, the third line L3 is indicated by a dash double-dot line. Note that the first line L1, the second line L2, and the third line L3 may be collectively referred to as lines L.

The controller 20 determines whether the order of the plurality of lines L is to be viewed clockwise or counterclockwise based on the inferred joint information. For example, the counterclockwise direction is associated with “right knee joint” of the joint information, and the clockwise direction is associated with “left knee joint” of the joint information. In the present embodiment, since the joint information is “right knee joint”, the order of the plurality of lines L is viewed counterclockwise. The controller 20 determines whether the first line L1, the second line L2, and the third line L3 are present in this order when viewed counterclockwise with reference to the first line L1 in the middle of FIG. 11A, (Step S31). In a case where it is determined that the condition of Step S31 is satisfied, the controller 20 proceeds to Step S32.

When the order is the first line L1, the second line L2, and the third line L3, the controller 20 determines that the femoral condyle portion is internally rotated (Step S32). That is, the controller 20 determines that the femoral condyle portion should be externally rotated in order to correct the positioning in the correct direction. Note that FIG. 11A is an example in which the femoral condyle portion of the radiation image G is externally rotated, and Step S32 does not correspond to FIG. 11A.

On the other hand, in a case where it is determined that the condition of Step S31 is not satisfied, the controller 20 proceeds to Step S33. The controller 20 determines whether the first line L1, the third line L3, and the second line L2 are present in this order when viewed counterclockwise with reference to the first line L1 in the middle of FIG. 11A (Step S33). If the controller 20 determines that the condition of Step S33 is satisfied, the controller 20 proceeds to Step S34.

When the order is the first line L1, the third line L3, and the second line L2, the controller 20 determines that the femoral condyle portion is externally rotated (Step S34). That is, the controller 20 determines that the femoral condyle portion should be internally rotated in order to correct the positioning in the correct direction. Note that FIG. 11A is an example in which the femoral condyle portion of the radiation image G is externally rotated, and Step S34 corresponds to FIG. 11A.

On the other hand, when determining that the condition of Step S33 is not satisfied, the controller 20 proceeds to Step S35. In this case, the controller 20 determines that the medial condyle side line information T1 and the lateral condyle side line information T2 of the femoral condyle portion overlap with each other and the femoral condyle portion is not misaligned (Step S35). Note that the determination that a misalignment of the femoral condyle portion does not occur is not limited to the case where the medial condyle side line information T1 and the lateral condyle side line information T2 completely overlap each other. When the overlap of the medial condyle side line information T1 and the lateral condyle side line information T2 is within an allowable range regarding the shift amount, it may be determined that a misalignment of the femoral condyle portion does not occur. When Step S35 ends, the controller 20 ends the subroutine of the first determination processing, and proceeds to Step S23 in FIG. 10.

Note that although the case of the right knee joint has been described in the first determination processing, the type of positioning misalignment can be determined by similar processing also in the case of the left knee joint or the like. In the case of the left knee joint, the intersections between the plurality of lines L radially extending from the femoral condyle portion center information C1 and the medial condyle side line information T1 and the like are viewed clockwise with respect to the radiation image G. When the order is the first line L1, the second line L2, and the third line L3, the controller 20 determines that the femoral condyle portion is internally rotated. When the order is the first line L1, the third line L3, and the second line L2, the controller 20 determines that the femoral condyle portion is externally rotated.

Subsequently, the controller 20 determines whether the misalignment is adduction or abduction based on the intersection pattern between the line connecting the femoral condyle center information C1 and the crural condyle center information C2 and the medial condyle side line information T1 or the like (Step S23). In the present embodiment, processing of determining whether the positioning misalignment is adduction or abduction is referred to as a second determination processing.

The controller 20 proceeds to subroutine shown in FIG. 13 and functions as the second determiner to execute the second determination process. FIG. 13 is a flowchart illustrating an example of the second determination processing.

As illustrated in FIG. 11B, the controller 20 connects the inferred femoral condyle center information C1 and the crural condyle center information C2 with a fourth line L4 (Step S40).

The controller 20 determines whether or not the fourth line L4 crosses the medial condyle side line information T1 and the lateral condyle side line information T2 in this order when the fourth line L4 is viewed from the femoral condyle center information C1 toward the crural condyle center information C2 (Step S41). In a case where it is determined that the condition of Step S41 is satisfied, the controller 20 proceeds to Step S42.

When the fourth line L4 intersects with the medial condyle side line information T1 and the lateral condyle side line information T2 in this order, the controller 20 determines that the femoral condyle part is adducted (Step S42). That is, the controller 20 determines that the femoral condyle portion should be abducted in order to correct the positioning in the correct direction. Note that FIG. 11B is an example in which the femoral condyle portion of the radiation image G is abducted, and Step S42 does not correspond to FIG. 11B.

On the other hand, when determining that the condition of Step S41 is not satisfied, the controller 20 proceeds to Step S43. The controller 20 determines whether or not the fourth line L4 intersects with the lateral condyle side line information T2 and the medial condyle side line information T1 in this order when viewing the fourth line L4 from the femoral condyle center information C1 toward the crural condyle center information C2 (Step S43). When the controller 20 determines that the condition of Step S43 is satisfied, the process proceeds to Step S44.

When the fourth line L4 intersects with the lateral condyle side line information T2 and the medial condyle side line information T1 in this order, the controller 20 determines that the femoral condyle portion is abducted (Step S44). That is, the controller 20 determines that the femoral condyle portion should be adducted in order to correct the positioning in the correct direction. Note that FIG. 11B is an example in which the femoral condyle portion of the radiation image G is abducted, and Step S44 corresponds to FIG. 11B.

On the other hand, when the controller 20 determines that the condition of Step S43 is not satisfied, the process proceeds to Step S45. In this case, the controller 20 determines that the medial condyle side line information T1 and the lateral condyle side line information T2 of the femoral condyle portion overlap with each other and no misalignment occurs (Step S45). The determination that no misalignment occurs also includes a case where the overlap between the medial condyle side line information T1 and the lateral condyle side line information T2 is within an allowable range regarding the shift amount. Note that Step S45 is processing common to Step S35 in FIG. 12, and thus may be omitted. When Step S45 ends, the controller 20 ends the subroutine of the second determination process, and proceeds to Step S24 in FIG. 10.

Note that in the second determination processing, the case where the imaging site is the lateral side of the right knee joint has been described, but also in the case where the imaging site is the lateral side of the left knee joint or the like, the type of positioning misalignment can be determined by similar processing. A specific determination method is common to the case of the lateral side of the right knee joint, and thus detailed description thereof will be omitted.

Further, whether or not the femoral condyle portion is adducted or the like is determined by using the fourth line L4 connecting the femoral condyle center information C1 and the crural condyle center information C2 shown in FIG. 11B, but the present invention is not limited thereto. The following determination method may be adopted. For example, a triangular virtual region V indicated by a broken line in FIG. 11B is assumed between the femoral condyle center information C1 and the crural condyle center information C2. Subsequently, a plurality of lines are drawn in the virtual region V in a direction from the femoral condyle center information C1 toward the crural condyle center information C2. At this time, in the plurality of lines, the number of lines intersecting in the order of the medial condyle side line information T1 and the lateral condyle side line information T2 is compared with the number of lines intersecting in the order of the lateral condyle side line information T2 and the medial condyle side line information T1. Finally, whether the femoral condyle portion is adducted or abducted may be determined based on which number is larger.

Next, the controller 20 derives the imaging support information I associated with the determination result of the positioning in Step S22 and Step S23 from the storage section 23 (Step S24). To be more specific, for example, when the result of the first determination processing indicates external rotation shown in FIG. 11A, the controller 20 refers to the imaging support information output table 23b and acquires the imaging support information I saying “please rotate the subject's knee internally”. Furthermore, for example, when the result of the second determination processing indicates abduction shown in FIG. 11B, the controller 20 refers to, for example, the imaging support information output table 23b and acquires the imaging support information I saying “please adduct the subject's knee”.

Note that the controller 20 may acquire command information indicating that the positioning is normal when the determination result indicates that there is no positioning misalignment in Step S22 and Step S23. When Step S24 ends, the controller 20 ends the subroutine and returns to Step S16 shown in FIG. 8.

The controller 20 performs display control to display the three-dimensional structure information T and the imaging support information I, which are the acquired determination results of the re-imaging determination processing, on the imaging screen 80 of the display part 22 (Step S16).

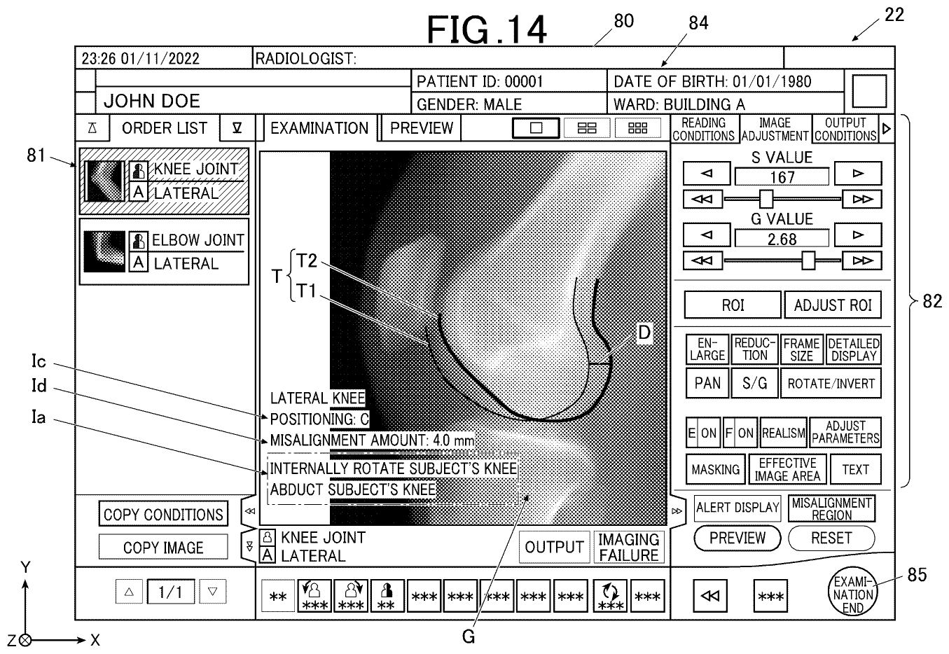

FIG. 14 shows an example of the three-dimensional structure information T and the imaging support information I displayed on the imaging screen 80. In addition to the radiation image G obtained by imaging, the three-dimensional structure information T is displayed in the image display area 83 of the imaging screen 80. The three-dimensional structure information T includes, in the femoral condyle portion of the radiation image G, the medial condyle side line information T1 indicating a medial condyle region and the lateral condyle side line information T2 indicating the lateral condyle region. The medial condyle side line information T1 and the lateral condyle side line information T2 which are correct answers in the femoral condyle portion are displayed in the image display area 83 of the imaging screen 80, superimposed on the radiation image G. The medial condyle side line information T1 and the lateral condyle side line information T2 may be displayed in different colors. To be specific, the medial condyle side line information T1 may be displayed in red (thin line in FIG. 14), and the lateral condyle side line information T2 may be displayed in blue (thick line in FIG. 14). In addition, the medial condyle side line information T1 and the lateral condyle side line information T2 may be displayed with different line thicknesses, or may be displayed with a solid line and a broken line in a distinguished manner. In order to realize these, correspondences between the “medial condyle side line information T1” and the “lateral condyle side line information T2” and the “color, thickness, line pattern (solid line, broken line)” are set in advance. The correspondences may be a default setting, or may be appropriately set by a user after shipment. The user can quickly specify the three-dimensional positional relationship between the medial condyle side line information T1 and the lateral condyle side line information T2 by memorizing the correspondences in advance or confirming a correspondence table or the like indicating the correspondences. The correspondence table or the like indicating the correspondences may be displayed on the imaging screen 80.

In addition to the radiation image G obtained by imaging, the imaging support information I based on the three-dimensional structure information T is displayed in the image display area 83 of the imaging screen 80. The imaging support information I is composed of, for example, words or sentences including technical terms. Specifically, when the direction of positioning correction is internal rotation, a sentence “Please internally rotate the subject's knee” is displayed as the imaging support information I in the image display area 83. For example, when the direction of positioning correction is adduction, a sentence “Please adduct the subject's knee” is displayed as the imaging support information I in the image display area 83.

Above the imaging support information I, rank information Ic and shift amount information Id are displayed. The rank information Ic and the shift amount information Id are information for alerting the user that the positioning needs to be corrected and presenting detailed correction contents. In the present embodiment, the imaging support information I and the like are displayed in an empty space without the radiation image G in the image display region 83, but the present invention is not limited thereto.

The rank information Ic is information indicating a degree of the shift amount of the positioning misalignment by a rank. For example, when there is no positioning misalignment and re-imaging is not required, “positioning: A” is displayed as the rank information Ic. When the shift amount of the positioning misalignment is within an allowable range and re-imaging is not required, “positioning: B” is displayed as the rank information Ic. When the shift amount of the positioning misalignment exceeds the allowable range and re-imaging is required, “positioning: C” is displayed as the rank information Ic. In FIG. 14, the case of “positioning: C” as the rank information Ic is illustrated.

The shift amount information Id is information indicating a distance (shift width) in a predetermined direction between the inferred medial condyle side line information T1 and the inferred lateral condyle side line information T2 of the femoral condyle portion. For example, when the length D in the X direction between the medial condyle side line information T1 and the lateral condyle side line information T2 is “4 mm”, “shift amount: 4.0 mm” is displayed in the image display area 83 as the deviation amount information Id. In FIG. 14, the shift amount in the X direction is displayed as the shift amount information Id, but the present invention is not limited thereto. For example, the shift amount in the Y direction may be displayed, or the shift amounts in both the X direction and the Y direction may be displayed.

The user confirms the three-dimensional structure information T and the imaging support information I displayed on the imaging screen 80 to guide the patient and correct the positioning misalignment. When the positioning misalignment is resolved, the radiation image is re-captured.

On the other hand, in a case where it is determined that there is no positioning misalignment, the controller 20 may display only the radiation image G in the image display area 83 of the imaging screen 80. In addition, in a case where it is determined that there is no positioning misalignment, the controller 20 may display “position: A” as the rank information Ic and “deviation amount: 0 mm” as the deviation amount information Id in the image display area 83. In addition, the controller 20 may display a sentence or the like indicating that there is no positioning misalignment in the image display area 83 as the imaging support information I.

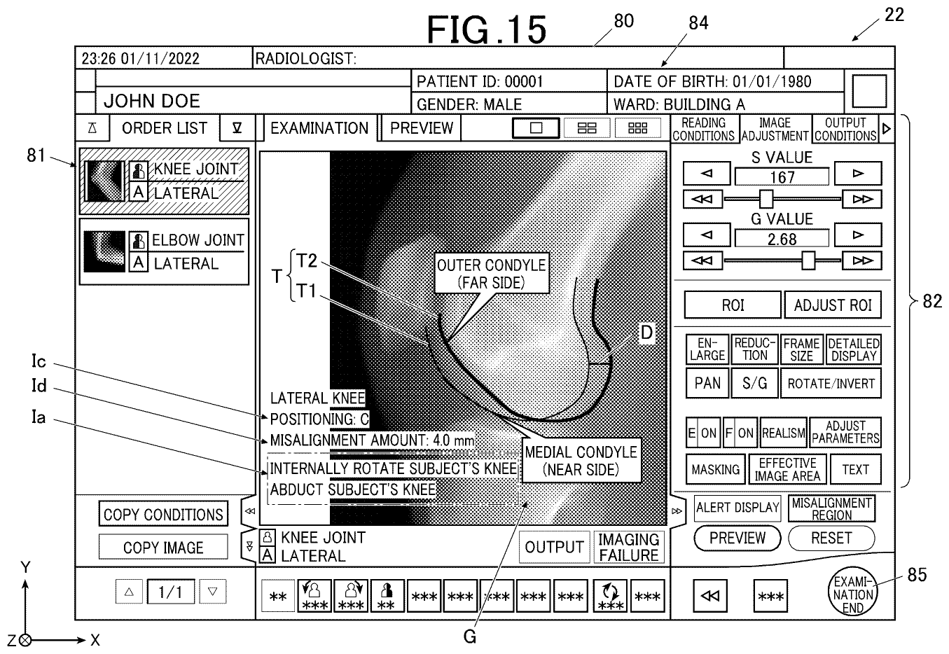

Furthermore, the method of displaying the medial condyle side line information T1 and the lateral condyle side line information T2 is not limited to the display method illustrated in FIG. 14. FIG. 15 shows an example of the imaging screen 80 in the case where text information is displayed in addition to each of the line information as the three-dimensional structure information T. In the image display area 83 of the imaging screen 80, the medial condyle side line information T1 indicating the medial condyle region of the femoral condyle and the lateral condyle side line information T2 are displayed as the three-dimensional structure information T, respectively. In the medial condyle side line information T1, text information of “medial condyle (near side)” indicating that the line information represents the medial condyle and is positioned on the near side is displayed in association. In the lateral condyle side line information T2, text information of “lateral condyle (far side)” indicating that the line information represents the medial condyle and is positioned on the far side is displayed in association. The display method of the character information is not limited to FIG. 14, and for example, only the character information of the “medial condyle” and the “lateral condyle” may be displayed.

Further, as a display method different from the three-dimensional structure information T described with reference to FIG. 14 and the like, for example, only one of the medial condyle side line information T1 and the lateral condyle side line information T2 may be superimposed on the “femoral condyle portion” of the radiation image G and displayed. This is because, depending on the user, if one of the medial condyle side line information T1 and the lateral condyle side line information T2 can be recognized, the other line information can be guessed.

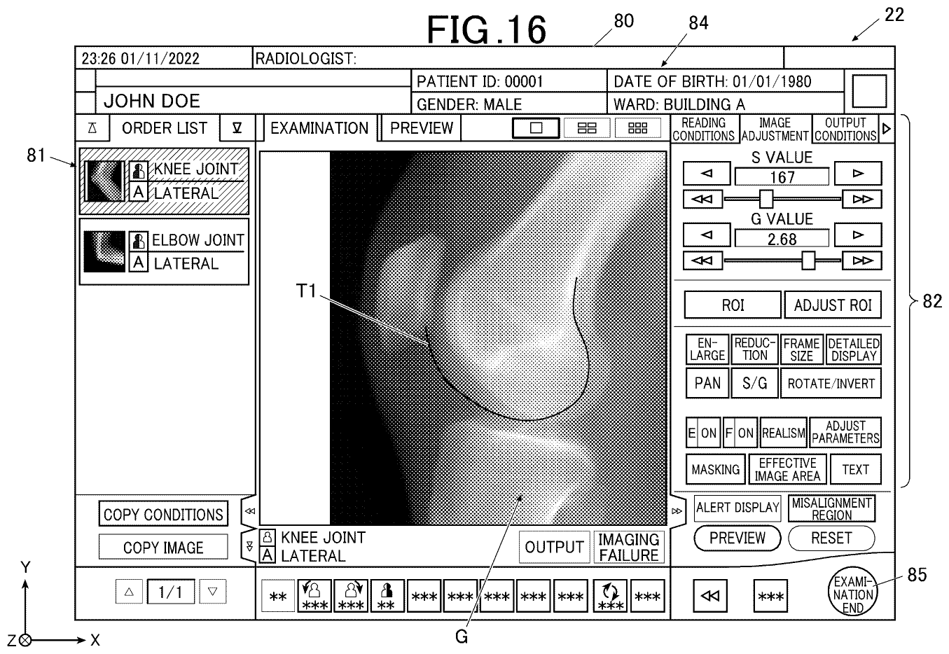

FIG. 16 shows an example of the imaging screen 80 in a case where only the medial condyle side line information T1 is displayed as the three-dimensional structure information T. In the image display area 83 of the imaging screen 80, the medial condyle side line information T1 indicating the medial condyle region of the femoral condyle is displayed as the three-dimensional structure information T, superimposed on the radiation image G. The medial condyle side line information T1 may be displayed with a color such as red, for example, similarly to FIG. 14. In a case where two lines indicating the bone ends can be recognized in the femoral condyle portion of the radiation image G, the line indicating one bone end can be specified as the medial condyle side line by displaying the medial condyle side line information T1 on the imaging screen 80. As a result, it is also possible to specify that the other remaining line indicating the bone end is the lateral condyle side line. Note that although the example in which the medial condyle side line information T1 is displayed as the three-dimensional structure information T is illustrated in FIG. 16, the lateral condyle side line information T2 may be displayed.

As described above, according to the present embodiment, the controller 20 infers the three-dimensional structure information T of the structure inside the subject in the radiation image. To be specific, by inputting a radiation image of the “lateral side of the right knee joint” to the machine learning model 23c, it is possible to perform inference while distinguishing between the medial condyle side line information T1 located on the near side and the lateral condyle side line information T2 located on the far side in the “femoral condyle portion”. A user such as a radiologist can specify a three-dimensional positional relationship in the “femoral condyle portion” by checking the medial condyle side line information T1 and the lateral condyle side line information T2 which are superimposed on the radiation image and displayed in the imaging screen 80. This allows the user to efficiently determine the direction of positioning correction. Furthermore, according to the present embodiment, since the imaging support information I is displayed on the imaging screen 80, the user can quickly determine the positioning by checking the imaging support information I. As a result, the speed of radiation imaging can be increased, and the burden on the patient during positioning can be reduced.

### Other Embodiment 1

In Other Embodiment 1, positioning support is performed using an optical camera image captured by an optical camera. Note that hereinafter, differences from the above-described embodiment will be mainly described, and description of points common to the above-described embodiment will be omitted. Furthermore, in the description of Other Embodiment 1, the same parts as those in the above-described embodiment will be described with the same reference symbols.

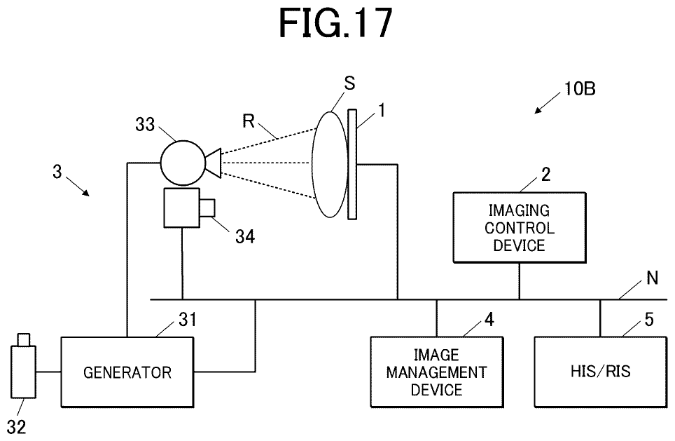

FIG. 17 is a diagram illustrating a schematic configuration of an imaging support system 10B according to Other Embodiment 1. The imaging support system 10B includes a radiographic imaging device 1, an imaging control device 2, a radiation generation device 3, an image management device 4, and a HIS/RIS 5. The generation device 3 includes a generator 31, a switch 32, a radiation source 33, and an optical camera 34. The optical camera 34 and the like are connected to the imaging control device 2 via a network N.

For example, the optical camera 34 is arranged side by side at a position adjacent to the radiation source 33. The radiation source 33 and the optical camera 34 may be integrally mounted in one housing. The optical camera 34 optically captures an image of the subject S at a timing before capturing a radiation image of the subject S. The optical camera 34 transmits the captured optical camera image corresponding to the positioning of the patient to the imaging control device 2. The optical camera image includes a still image or continuously captured dynamic images.

The imaging control device 2 determines the presence or absence, the type, and the like of the positioning misalignment based on the optical camera image acquired from the optical camera 34. To be specific, the imaging control device 2 infers the three-dimensional structure information T such as the medial condyle side line information T1 by using the machine learning model 23c. The imaging control device 2 specifies the type of deviation in positioning based on the three-dimensional structure information T or the like, and acquires the imaging support information I corresponding to the type of misalignment. The imaging control device 2 displays the imaging support information I, the three-dimensional structure information T, and the like on, for example, the display device 26 and the display part 22. A user such as a radiologist can recognize the three-dimensional positional relationship of the misalignment region from the three-dimensional structure information T, and can easily understand the direction of positioning correction from the imaging support information I. In a case where the positioning misalignment is resolved, the user proceeds to the next step, that is, imaging of a radiation image of the subject S.

According to Other Embodiment 1, similarly to the above-described embodiment, the user can specify the three-dimensional positional relationship in the “femoral condyle portion” by checking the medial condyle side line information T1 and the lateral condyle side line information T2 which are superimposed on the radiation image and displayed in the imaging screen 80. This allows the user to efficiently determine the direction of positioning correction. Further, according to the present embodiment, since the imaging support information I is displayed on the imaging screen 80, the user can quickly determine the positioning by checking the imaging support information I. As a result, the speed of radiation imaging can be increased, and the burden on the patient during positioning can be reduced. Furthermore, by using the optical camera 34, it is possible to correct positioning before radiation imaging. Thus, the number of times of re-imaging can be reduced, and the burden on the patient can also be reduced by reducing the total amount of exposure of the patient.

Although the preferred embodiments of the present disclosure have been described in detail with reference to the accompanying drawings, the technical scope of the present disclosure is not limited to such examples. Furthermore, those to which various modification examples and improvements have been applied naturally belong to the technical scope of the present disclosure within the category of the technical idea described in the scope of the claims of those skilled in the art.

In the above-described embodiment, the three-dimensional structure information T of the “femoral condyle portion” is inferred in a case where the radiation image is of the lateral side of the knee joint. However, the imaging site or the like to be inferred is not limited to the lateral side of the knee joint. For example, even in a case where the radiation image is of the lateral side of the ankle joint, the lateral side of the knee joint, or the like, the three-dimensional structure information T of the structure(s) inside a subject can be estimated by applying the above-described re-imaging determination processing.

1. An image processing apparatus comprising:
a hardware processor,
wherein the hardware processor acquires a two-dimensional radiation image; and
analyzes the acquired two-dimensional radiation image to infer three-dimensional structure information on a structure on an inside of a subject.

2. The image processing apparatus according to claim 1,
wherein the hardware processor outputs the inferred three-dimensional structure information.

3. The image processing apparatus according to claim 1,
wherein the hardware processor infers, as the three-dimensional structure information on the inside of the subject, information on a position in an irradiation direction.

4. The image processing apparatus according to claim 1,
wherein the hardware processor infers, as the three-dimensional structure information on the inside of the subject, information for distinguishing between a structure on a near side and a structure on a far side in an irradiation direction.

5. The image processing apparatus according to claim 1,
wherein the hardware processor extracts the structure on the inside of the subject from the two-dimensional radiation image,
wherein the hardware processor infers, as the three-dimensional structure information, three-dimensional structure information on the extracted structure.

6. The image processing apparatus according to claim 5,
wherein the hardware processor acquires imaging site information,
wherein the hardware processor extracts a structure of the subject from the two-dimensional radiation image based on the acquired imaging site information.

7. The image processing apparatus according to claim 1,
wherein the three-dimensional structure information inferred by the hardware processor includes information of a medial condyle side line and information of a lateral condyle side line of a femoral condyle.

8. The image processing apparatus according to claim 2,
wherein the hardware processor displays the inferred three-dimensional structure information superimposed on the two-dimensional radiation image.

9. The image processing apparatus according to claim 1, further comprising:
a re-imaging support information output section that outputs re-imaging support information based on the three-dimensional structure information inferred by the hardware processor.

10. The image processing apparatus according to claim 9,
wherein the re-imaging support information is information for changing a position of the subject or an imaging device at a time of re-imaging.

11. The image processing apparatus according to claim 1,
wherein the hardware processor inputs, in a machine learning model which has learned radiation images including a structure inside a subject as input data and three-dimensional structure information on the structure inside the subject as correct output data, the radiation image acquired by the hardware processor to infer the three-dimensional structure information of the structure in the radiation image.

12. A non-transitory computer-readable storage medium storing a program for causing a computer to function as:
a first acquisition section that acquires a two-dimensional radiation image; and
an inference section that analyzes the acquired two-dimensional radiation image to infer three-dimensional structure information on a structure on an inside of a subject.

13. The storage medium according to claim 12,
wherein the program causes the computer to function as:
an output section that outputs the inferred three-dimensional structure information.

14. The storage medium according to claim 12,
wherein the inference section infers, as the three-dimensional structure information on the inside of the subject, information on a position in an irradiation direction.

15. The storage medium according to claim 12,
wherein the inference section infers, as the three-dimensional structure information on the inside of the subject, information for distinguishing between a structure on a near side and a structure on a far side in an irradiation direction.

16. The storage medium according to claim 12,
wherein the program causes the computer to function as:
an extraction section that extracts the structure on the inside of the subject from the two-dimensional radiation image,
wherein the inference section infers, as the three-dimensional structure information, three-dimensional structure information on the extracted structure.

17. The storage medium according to claim 16,
wherein the program causes the computer to function as:
a second acquisition section that acquires imaging site information,
wherein the extraction section extracts a structure of the subject from the two-dimensional radiation image based on the acquired imaging site information.

18. The storage medium according to claim 12,
wherein the three-dimensional structure information inferred by the inference section includes information of a medial condyle side line and information of a lateral condyle side line of a femoral condyle.

19. The storage medium according to claim 13,
wherein the output section displays the inferred three-dimensional structure information superimposed on the two-dimensional radiation image.

20. The storage medium according to claim 12,
wherein the program causes the computer to function as:
a re-imaging support information output section that outputs re-imaging support information based on the three-dimensional structure information inferred by the inference section.

21. The storage medium according to claim 20,
wherein the re-imaging support information is information for changing a position of the subject or an imaging device at a time of re-imaging.

22. An image processing method comprising:
acquiring a two-dimensional radiation image; and
analyzing the acquired two-dimensional radiation image to infer three-dimensional structure information on a structure on an inside of a subject.
